(19) United States
(12) Patent Application Publication
(10) Pub. No.: US 2025/0155431 A1
(43) Pub. Date: May 15, 2025

(54) MACROSCOPIC LASER-INDUCED GRAPHENE

(71) Applicant: B.G. NEGEV TECHNOLOGIES AND APPLICATIONS LTD., AT BEN-GURION UNIVERSITY, Beer Sheva (IL)

(72) Inventors: Christopher ARNUSCH, Jerusalem (IL); Assaf YA’AKOBOVITZ, Ra’anana (IL); Chetan Prakash SHARMA, Midreshet Ben-Gurion (IL); Yahav BEN-SHIMON, Ashdod (IL)

(73) Assignee: B.G. NEGEV TECHNOLOGIES AND APPLICATIONS LTD., AT BEN-GURION UNIVERSITY, Beer Sheva (IL)

(21) Appl. No.: 18/726,937

(22) PCT Filed: Jan. 6, 2023
(86) PCT No.: PCT/IL2023/050021

§ 371 Date: Jul. 5, 2024

Related U.S. Application Data
(60) Provisional application No. 63/296,889, filed on Jan. 6, 2022.
(60) Provisional application No. 63/337,191, filed on May 2, 2022.

(51) Int. Cl. G01N 33/543; C01B 32/184; G01N 29/12; G01N 33/569

(52) U.S. Cl. CPC G01N 33/54373; C01B 32/184; G01N 29/12; G01N 33/56983; C01B 2204/04; C01B 2204/32; C01P 2002/82; C01P 2004/03; C01P 2004/20; G01N 2291/014; G01N 2333/165; G01N 2469/10

(57) ABSTRACT

Provided herein macroscopic laser-induced graphene flakes and sheets, methods of manufacturing same, and the use of same in diagnostic and other applications.

### FIELD OF THE INVENTION

The present disclosure relates to a new physical form of laser-induced graphene as macroscopic flakes and/or continuous sheets, methods of manufacturing same, and use thereof in a variety of applications, e.g., in sensors.

### BACKGROUND

Laser-induced graphene (LIG) is a specific three-dimensional porous form of graphene (e.g., graphene foam) prepared by laser irradiation of a variety of substrates, notably polymers, polyimide in particular. This form, including the manufacturing and various uses thereof have been disclosed, inter alia, in PCT publications of patent applications WO2015175060, WO2017223217, WO2018085789, and WO2017199247; in particular, PCT patent application WO2015175060 discloses laser induced graphene (LIG) materials and their use in electronic devices; and PCT patent application WO2018085789 discloses further methods of fabricating LIG and compositions thereof, including fabricating LIG on surfaces of various polymers, including polysulfone, polyethersulfone, and polyphenylsulfone sheets. Using LIG in sensing applications has also been disclosed, inter alia in WO2020197606. In the latter publication, the obtained patterned graphene foam has been transferred from the polymer wherein it had been formed on an adsorption carrier.

LIG has also been manufactured in form of microscopic fibers, with the length of about 1 mm and up to several microns thickness, as disclosed in L. X. Duy et al., Carbon 126 (2018) 472-479. Manufacturing of macroscopic standalone flakes of graphene foam sheets, including those similar to LIG, while theoretically possible, has been limited by complexity of the techniques required, e.g., chemical vapor deposition on porous substrate, followed by meticulous etching and drying, or via graphene oxide intermediates, and as a consequence is not scalable. As to LIG, after lasing, LIG is usually embedded in the substrate material. LIG honeycombs, dubbed “honeycomb porous graphene”, have been reported by Xu et al in ACS Nano 2021, 15, 8907-8918; they are also embedded in a polymeric substrate. If required, graphenic material may be separated from the substrate by further steps, usually mechanical, that remove the LIG material from the substrate. This may produce LIG that is separated from the substrate, but methods that might be used for this purpose, for example scraping, will destroy the extended continuous graphene foam sheet structure obtained on the substrate and will not furnish any macroscopic-sized flakes.

Therefore, there is a need in the art to provide standalone LIG graphene foam sheets with a continuous structure.

### SUMMARY OF THE INVENTION

It has now been unexpectedly found that using the laser ablation technique on an amenable polymer at a specific energy output, it may be possible to prepare readily detachable, macroscopic standalone LIG graphene foam flakes, e.g., LIG graphene foam material that is unsupported on a substrate, as opposed to graphene-coated or graphene-scribed substrates. The resultant product has dimensions in millimeter to centimeter range, although the thickness usually remains in the order of magnitude of tens of microns, but suitable mechanically integral pieces of the material (i.e., flakes) may be obtained. Additionally, mechanically integral sheets having a plan area which is generally believed to be limited only by the irradiation area, may also be obtained, using similar parameters. Pieces of any suitable form or size may then be prepared from these sheets and/or flakes. These may have a variety of applications, including in micro- and macro-composite materials, for electromagnetic interference shielding, Joule heating, and may also be used as cantilevers for a variety of applications, e.g., sensing applications. As this material can be readily chemically functionalized, it can be used for specific sensing of chemical and biological agents, e.g., as resonating cantilevers. In their turn, the resonating cantilevers (resonators) can be used in a variety of applications, whereof sensing is only one of preferred functions. As demonstrated in the appended examples, functionalization of the freestanding LIG flakes with antibodies directed to the spike protein of COVID-SARS-2 virus can detect and even quantify the spike protein of the COVID-19 coronavirus with a limit of quantification of as low as merely about five thousand virions.

Therefore, provided herein a standalone macroscopic graphene foam sheet flake, wherein said graphene foam is laser-induced graphene, wherein said standalone sheet flake is not supported on a carrier or embedded in a polymer, wherein said standalone sheet flake is mechanically integral, and wherein said flake has a thickness of between 5 and 200 microns. The flake may preferably be characterized by a porous stochastic morphology on one side, and on the opposing side a honeycomb-like cellular structure of a plurality of interconnected cells having a plan projection of a symmetric or asymmetric polygon having between 3 and 10 sides, as seen under suitable magnification using scanning electron microscopy (SEM). The honeycomb-like cellular structure may have an overall appearance of co-oriented fused LIG continuous flake (LIGCF) fibers. Alternatively, the honeycomb-like cellular structure may have an overall appearance of an essentially uniform worm-like patterned LIGCF sheet with randomly oriented interfused LIGCF fibers. The flake may be wherein said thickness is between 20 and 35 microns. The flake may also be wherein said macroscopic flake has a length of at least 1 millimeter. Preferably, the flake may be wherein said length of at least 10 millimeters. The flake may also be wherein said flake is mechanically integral over an area of between 1 square centimeter and 1,000 square centimeters. The flake may be further comprising a targeting moiety associated therewith. Preferably, the flake may be wherein said targeting moiety is an anti-SARS-CoV-2 spike receptor-binding domain antibody. In a further aspect provided herein a resonating sensor comprising a standalone macroscopic graphene foam sheet flake as generally described herein. In a further aspect, provided herein a method of manufacturing of standalone macroscopic graphene foam sheet flakes as described herein, the method comprising providing a sheet of a polymer, and irradiating said sheet of polymer with laser having power or energy density equivalent to that obtainable with laser power output of 4.5 to 9 W from a laser equipped with a lens providing a pulse diameter of 130 microns at 1000 pulses per inch density, wherein a focal offset of said laser is between about 100.2% and 101.2% of the focal length of said laser lens, or between 98% and 90% of the focal length of said lens, and wherein a minimum irradiation area of said polymer between 1 and 4 square centimeters. The method is preferably wherein said polymer is a polyimide. The method may further preferably be wherein said irradiation area is between 1 square centimeter and 1 square meter. The method may further preferably be wherein said focal offset of said laser is 90 and 98% of said focal length of said lens, and wherein said irradiation area is between 40 and 200 square centimeters.

### BRIEF DESCRIPTION OF FIGURES

FIG. 1 presents a photograph of the substrate after one cycle of laser treatment that shows LIG flakes formation.

FIG. 2 presents a photograph of two freestanding LIG sheets as obtained according to a procedure in the Example 3, having the mechanically integral area of about 50 square centimeters (left sheet, partially rolled) and 96 square centimeters (right sheet).

FIG. 3 presents inlaid scanning electron micrographs of LIGCF according to the example 2.

FIG. 4 presents inlaid scanning electron micrographs of extra-large LIG sheets according to the example 3.

FIG. 5 represents Raman spectroscopic analysis for freestanding LIG flakes of the Example 2.

FIG. 6 represents Raman spectroscopy of the extra large freestanding LIG sheets according to Example 3 at various offset settings.

FIG. 7 represents Raman spectroscopic analysis for freestanding LIG flakes obtained at 12% power output.

FIG. 8 demonstrates frequency response for LIGCF cantilever resonator sensor.

FIG. 9 demonstrates the change in the resonance frequency of the sensor according to an embodiment of the present invention, responsive to binding of a target moiety.

FIG. 10 demonstrates a chart detecting added mass as function of frequency shift observed, according to an embodiment of the invention.

FIG. 11 demonstrates the greyscale thermal images series captured by IR camera during Joule heating according to the Example 8.

FIG. 12 demonstrates scanning electron micrograph of LIG sheet laminates with improved mechanical properties according to the example 9.

### DETAILED DESCRIPTION

Thus, in one aspect, provided herein a standalone macroscopic graphene foam sheet flake. The flake is standalone, i.e., not supported on a substrate or carrier, and is not embedded into a substrate or a carrier, e.g., in a polymer. The flake is macroscopic, e.g., it has dimensions as generally described herein and below in particular, the dimensions being more than 1 millimeter, and may generally be only limited by the designed irradiation area. The terms “graphene foam”, “LIG”, and like, are generally used interchangeably in the present disclosure, unless the context clearly dictates otherwise. Generally, LIG is a single or few-sheet of a polycrystalline carbon layer(s), e.g. less than 10 layers, with atoms arranged in multiple polygon configurations, e.g. pentagon, hexagon and heptagon structures, which is in contrast to “classic” graphene consisting exclusively of sp²-hybridized carbon hexagons. Therefore, the terms “laser-induced graphene” and/or “LIG” encompass molecules structured into polycrystalline turbostratic carbon layers, arranged in pentagon, hexagon and heptagon configurations, in any shape or morphology. The term “graphene foam sheet”, as used herein, refers to continuous mechanically integral essentially planar (i.e., relatively thin) structures made of graphene foam, e.g., of LIG. Therefore, “graphene foam sheet flake” is a composition of matter in the form of a flake or a chip, e.g., a macroscopic piece of the sheet, being either physically excised, broken off, or otherwise derived from a larger freestanding sheet of graphene foam, such as LIG sheet.

The obtained LIG flakes and sheets have macroscopic dimensions, i.e., inter alia, they are characterized by a substantial mechanical integrity. They are also characterized by a continuity in at least one dimension (that is generally not thickness) of at least 1 mm, e.g., at least 2 mm, and may be even over 1 cm, and therefore they are termed “continuous”. Likewise, continuous standalone sheets may have almost any suitable shape and dimensions, generally dictated by the initial irradiation area, as elaborated below. That is, the standalone macroscopic LIG continuous flakes (as referred to herein as LIGCF) is an essentially homogenous graphene foam sheet, which when received, may be interrupted by natural cracks in the sheet, or may be further broken down by mechanical means to the needed dimensions. Moreover, as demonstrated in the appended examples, by utilizing the methodology as described herein unsupported continuous sheets, literally, of freestanding graphene foam of almost any desirable size can be obtained, depending on the lasing area, i.e., at least 1 cm, or at least 4 cm. as demonstrated in the appended examples, sheets of 8×12 cm (96 square centimeters!) were readily prepared and handled. However, in currently preferred embodiments, the thickness of LIGCF and of the sheets wherefrom they originate, may be of between about 5 microns to several hundreds of microns, e.g. between 5 and 100-300 microns, preferably between 15 microns to 40 microns, further preferably between 20 and 40 microns, e.g., with an average thickness of between 25 and 30 microns. The LIGCF are essentially homogenous and usually mechanically integral. They may have a length and/or width dimensions of up to coextensive with the original lasing pattern, or a smaller suitable size, but larger than 1 mm, e.g., larger than 2 mm, or even larger than 1 cm, as discussed in greater detail below. When referring to the axial dimensions of LIGCF as described herein, the thickness is usually the dimension perpendicular to the surface of the polymer sheet wherefrom the LIGCF is obtained (and consequently to the surface of the flake itself), the length is the longer dimension, and the width is the shorter dimension.

The LIGCF sheet is essentially homogenous and/or mechanically integral, with these terms being used herein interchangeably referring to the sheet that has a uniform structural pattern and/or other characteristics along at least one of length or width dimensions, or both, but may have a different structure in the thickness dimensions, as discussed in greater detail below. The essentially homogenous graphene foam sheets are usually characterized by homogenous effective properties, such as mechanical, electrical, thermal, and/or other physical properties. The essentially homogenous graphene foam sheets are preferably also characterized by mechanical integrity within the sheet and/or the flake. Some of the LIGCF may be regarded as rather long “flat fibers”, i.e., one dimension (“length”) is significantly larger than the others (usually “width”, and of course larger than “thickness”). The length of the LIGCF as obtained may be at least 2 times larger than the width, such as between 2 and 1000 times larger, preferably between 2 and 100 times larger. The length of the LIGCF after processing may between 2 and 50 times larger, preferably between 2 and 10 times larger than the width. Conversely, the width and thickness of the final LIGCF may even be of comparable dimensions, as in the case of sheets, and as may be required by the needs of the final application of LIGCF. Generally, the length of the LIGCF may be as large as the length of the lasing pattern, but preferably, for the sake of applications as resonating cantilever beam, the LIGCF have the length of between 1 and 50 mm, preferably between 1 and 5 mm.

The graphene foam sheets may usually be characterized by essentially homogenous mechanical integrity over the area limited only by the initial laser-irradiated area. The mechanically integral graphene foam sheet flakes may have an area of over 1 square centimeters, e.g., between 1 and 1,000 square centimeters, such as between 1 and 100 square centimeters. Depending on the equipment used, the mechanically integral graphene foam sheets may have a larger area, e.g., over 1 square meter.

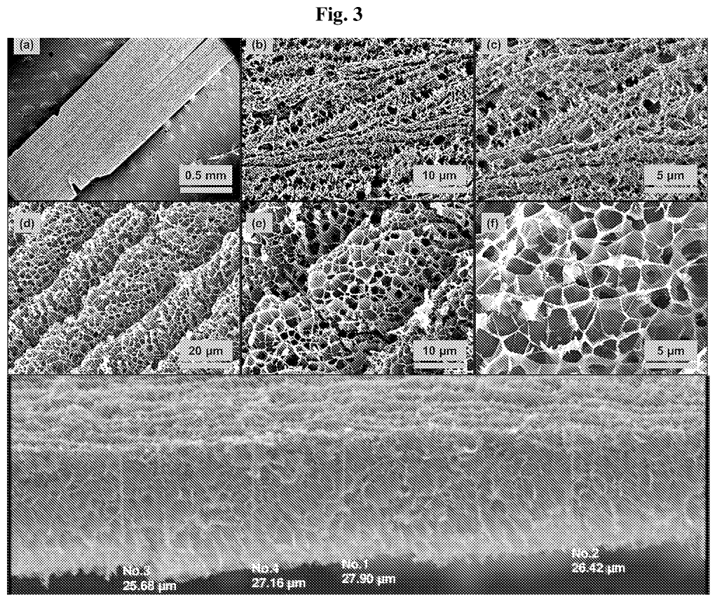

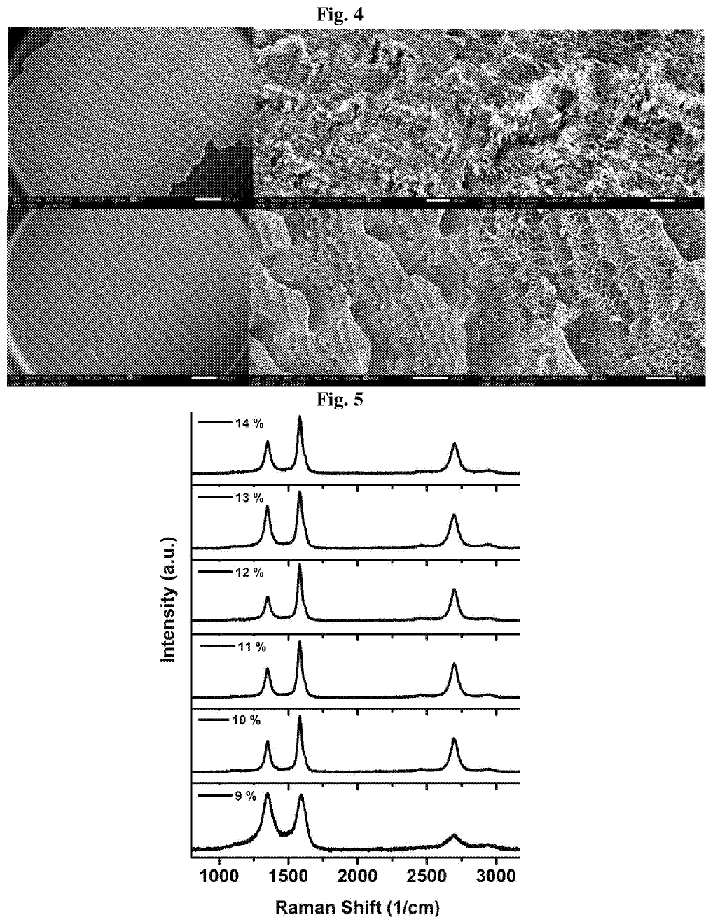

Additionally, the sheet may also be characterized by a certain degree of anisotropy in the thickness dimension—the surface that was facing the bottom of the polymer sheet (i.e. the “bottom” of LIGCF) may have a honeycomb microstructure, while the opposite surface (i.e., “top”, the surface that the laser was applied on) has a structure similar to observed with a known embedded LIG, i.e. porous stochastic morphology. In currently preferred embodiments, the bottom surface is usually more structured and is characterized by the honeycomb-like structured pattern with pores of size of about 4 microns, e.g., between 2 and 8 microns. The term “honeycomb” is used herein descriptively and does not imply a strict symmetric hexagonal repeating pattern, but rather a honeycomb-like cellular structure of a plurality of interconnected cells having a plan projection of a symmetric or asymmetric polygon having between 3 and 10 sides. The pattern is best visualized when seen under suitable magnification, e.g., using scanning electron microscopy (SEM). Additionally, the top side may have an overall “macroscopic” appearance, i.e., as seen at an intermediate scale between the high magnification demonstrating the honeycomb polygons and the low magnification, may in some instances resemble the morphology of the “parent” polymer, whereas the bottom side may have either a co-oriented fused LIGCF fibers (e.g., as seen in the appended examples, spaced about 10 to 30, e.g., about 20 microns apart), or an essentially uniform worm-like patterned LIGCF with randomly oriented interfused LIGCF fibers. Generally, the exemplary microscopic patterns of the LIG continuous flakes and sheets are presented in the FIGS. 3 and 4. Without being bound by a particular theory it is currently believed that under the specific manufacturing conditions, as described below, the co-oriented or randomly diffused interfusion of LIG “filaments” provides sufficient mechanical integrity to detach from the substrate and withstand the handling of the sheets and flakes.

The macroscopic mechanically integral LIGCF and sheets, as generally described herein, constitute one aspect of the invention. In further aspect, provided utilizing the macroscopic mechanically integral LIGCF and sheets in a variety of applications, including in micro- and macro-composite materials, for electromagnetic interference shielding, Joule heating, filtration applications, as an electrical conductor, or resistor, or capacitor, and may also be used as cantilevers for a variety of applications, e.g., sensing applications. Specifically, the macroscopic mechanically integral LIGCF and sheets may be used in manufacturing of micro- and macro-composite materials, electromagnetic interference shielding, Joule heating, filtration systems, electrical conductors, resistors, or capacitors, and cantilevers for a variety of applications, e.g., sensing applications. Therefore, as a further aspect, provided herein any one of micro- and macro-composite materials, electromagnetic interference shielding material, Joule heaters, filtration systems, electrical conductors, resistors, or capacitors, and a cantilever apparatuses, each individually comprising macroscopic mechanically integral LIGCF and sheets.

The LIGCF may be functionalized for use in sensors, as described in further detail below. Functionalization of the LIGCF may be performed by any suitable method readily known in the art, whereby the surface of the LIGCF becomes coated with a targeting moiety. The targeting moiety may be directly bound to the LIGCF, or may be bound via a cascade of intermediate molecules, facilitating or enabling the binding of the targeting moiety onto the graphene foam sheet surface. The LIGCF coated with a targeting moiety may also be coated with a binding-blocking moieties, to reduce non-specific binding, e.g. to the uncoated portions of the LIGCF. Thus, in a separate aspect of the invention provided herein a macroscopic LIG continuous flake, further comprising a targeting moiety associated therewith, e.g., coated with the targeting moiety, and optionally with a blocking moiety to decrease or eliminate non-specific binding, as generally described herein. As described below, the targeting moiety may be an anti-SARS-CoV-2 spike receptor-binding domain antibody, e.g., to facilitate detection and optionally quantification of COVID-19 pathogens.

Sensors comprising LIGCF, coated or uncoated, may be configured in any suitable configuration as known in the art, for example, as described in Adv. Mater. 2021, 2101326, DOI: 10.1002/adma.202101326, or in J. Zhang and K. Hoshino, Molecular Sensors and Nanodevices, 2014, Elsevier Inc. More specifically, however, LIGCF may be used in resonating sensors. For example, graphene foam sheet may be suitably shaped into a LIGCF of desired dimensions, e.g., in a form of a beam or a slab. The flake may be suspended as cantilever from a conductive surface, connected to a stationary electrode. The LIGCF is thus moveable responsive to voltage change between the electrodes, as the induced electrostatic force actuates the suspended LIGCF. The resonance frequency may then be determined via a suitable optical means, e.g., laser Doppler vibrometer. Thus, in a further aspect, provided herein a resonating sensor comprising a graphene foam sheet flake, as generally described herein.

LIGCF and sheets may be fabricated, for example, on Kapton®, a polyimide (PI) (poly-(4,4′-oxydiphenylene-pyromellitimide)), or on poly (ether imides), or on other suitable polymer, as known in the art, e.g. other polyimides, polysulfones, polyethersulfones, polyphenylsulfones, or polyamide. The polymers may preferably be provided in the form of films or sheets. Then, the polymer surface may be exposed to laser. The exposing step may be conducted with a CO₂ laser, e.g., disposed in a cutter system, such as for example, Universal X-660 laser cutter platform, e.g. XLS10MWH, or a Universal Laser VLS3.50. At the parameters as described below, the polymer, e.g. PI, can be converted into LIG flakes and even continuous sheets. The exposure to laser may be performed under different gases' atmosphere, based on a gas box design, such as without being limited to, 100% air, or under hydrogen (H₂), argon (Ar), nitrogen (N₂), or oxygen (O₂) atmosphere. Preferably, the LIGCF are manufactured at ambient atmosphere. The laser lens may be kept clean by blowing the same gas, or air, onto it, to clear the debris and/or to cool it.

By a way of a non-limiting example, when the polymer is a polyimide and the laser system is a 50 W 10.6 μm CO₂ pulsed laser equipped with “2 inch” (50.80 mm) focal length laser lens that is characterized to produce a laser focal spot size of ca. 130 microns in diameter, LIGCF standalone flakes may be obtained with a process comprising laser irradiating of the substrate, with the laser power and/or energy density equivalent to obtainable with laser power setting of between 9 and 18% (of 50 W laser), which corresponds to 4.5 and 9 W, at 1000 pulses per inch (PPI), and at minimum irradiation area of between 1 and 4 cm². The substrate may be offset at a distance of between −0.7 mm to −0.2 mm of platform vertical shift from its home (i.e., zero) position, equal to an offset of the focal length of the laser between about 0.1 mm to about 0.6 mm away from the laser respectively, which is between about 100.2% and about 101.2% of the focal length of the laser lens. The platform shift values correspond to the conventional laser setup, wherein laser head is displaceable in a defined plane, and the substrate support platform is vertically displaceable from the focal length, taken as the zero. Therefore, negative numbers indicate lowering the platform, i.e. moving it away from the laser, whereas positive numbers indicate raising the platform towards the laser, from the zero distance. Additionally, LIGCF of particularly large dimensions with micro-honeycomb diffused porous morphology with randomly oriented interfused LIGCF fibers at the bottom may be obtained by moving the substrate towards the lens, with an offset of +1.0 mm to +5.0 mm, preferably between +1.0 and 3.8 mm, being equal to about 98% and 90% of focal distance of the laser lens. It is evident that other parameters may be used as available according to the specific laser apparatus used, e.g., by adjusting the laser spot diameter, e.g., between 100 and 160 microns, pulse density, e.g. between 600 and 1200 PPI, and the energy, provided that they correspond to a similar power or energy density and/or fluence, and the lased area. Typical laser machine parameters can include but not limited to power, speed, image density, PPI, and raster/vector modes.

The specific laser powers can vary according to the duty cycles used. For example, one can use a 50 W laser at 9% to 18% power, or duty cycle, meaning that the laser is “on” only 9% to 18% of the time, respectively. Similarly, when a 75 W laser is used, the duty cycle of between 6.75% and 13.5% may be used, meaning that the laser is “on” only 6.75% to 13.5% of the time, respectively, to provide the same power of between 4.5 and 9 W. Thus, the duty cycle depends on the wattage of the laser used and also the fluence or the step size between the laser pulses as it traverses across the polymer (e.g. the pulses density per area, and the rastering speed), e.g. PI substrate, producing LIG, depending on the fluence.

Thus, in a further aspect provided herein a method of manufacturing of macroscopic standalone LIG continuous flakes. The method may comprise providing a sheet of a polymer, preferably a polyimide, and irradiating it with laser having power or energy density equivalent to obtainable with laser power output of 4.5 to 9 W, having a pulse diameter of 130 microns, at 1000 ppi density, with focal offset between about 0.1 mm to 0.6 mm away from the laser respectively, which is between about 100.2% and 101.2% of the focal length of the laser lens, and with a minimum irradiation area being between 1 and 4 square centimeters, whereas total irradiation area being between 1 square centimeter and the area of the polymer sheet used. Alternatively, particularly when large LIG sheets are sought, the method may comprise providing a sheet of a polymer, preferably a polyimide, and irradiating it with laser having power or energy density equivalent to obtainable with laser power output of 4.5 to 9 W, having a pulse diameter of 130 microns, at 1000 ppi density, with focal offset between about 1.0 mm to +5.0 mm, preferably between +1.0 and 3.8 mm, being equal to about 98% and 90% of the focal length of the laser lens, and with a minimum irradiation area being between 1 and 4 square centimeters, whereas total irradiation area being between 1 square centimeter and the area of the polymer sheet used, e.g., 1 square meter, or 1,000, 800, 600, 400, or 200 square centimeters.

The obtained LIGCF readily detach from the polymer sheet and may be harvested, and further processed according to the need, e.g. trimmed, functionalized, ground, analyzed, etc.

Generally, the obtained LIG continuous flake or sheet material may comprise above 70% of carbon, e.g. above 85%, above 87%, or above 90%, and may also comprise oxygen and/or nitrogen, as long as this does not impair electrical or thermal conductivity. The LIG may have a minimum electrical conductivity of at least below 500 ohm/square sheet resistance, preferably below 100 ohm/square sheet resistance, and most preferably between 50 and 1.5 ohm/square sheet resistance.

Characterization of LIG may be performed by a scanning electron microscope (SEM, such as for example FEI Quanta 400 high resolution field emission instrument, or Jeol IT200 SEM), by a transmission electron microscope (TEM, such as for example 80 KeV JEOL ARM200F), by an X-ray photoelectron spectroscopy (XPS, such as for example PHI Quantera), by Raman spectroscopy (Raman, such as Horiba LabRam HR evolution micro-Raman system), and by a Fourier transform infrared spectroscopy (FT-IR, such as for example Nicolet infrared spectroscope), as known in the art.

The harvested LIGCF material may be used as is, e.g., in composite materials, or filtration applications, or as an electrical conductor, or resistor, or capacitor, as described generally above. The obtained LIGCF can also be functionalized using targeting moieties. The LIG continuous flake may be functionalized providing a further advantage of sensing specific molecules, such as biomolecules, nucleic acids, viral particles and/or proteins. The targeting moieties are usually characterized by a specific affinity to a target molecule. The targeting moieties may include antibodies or antibody fragments, other proteins or peptides having a specific affinity to any specific molecule (e.g. ligands to a receptor or an enzyme), saccharides capable of specifically interacting with a specific molecule. The targeting moieties may also be oligonucleotides of any suitable nucleic acid sequences, either DNA, RNA, or artificial nucleotides' analogs. Targeting moieties may also include non-specific moieties interacting with certain classes of compounds. In some preferred embodiments, the targeting moiety is an antibody. The antibody may have an Fc region bindable to the LIGCF, either directly or via a linking moiety, as described below, and a Fab region comprising at least one complementarity determining regions (CDR), which are capable of specific interaction with a target molecule. Alternatively, the antibody may be covalently bound to a linking moiety in a random position on the antibody, and this linking moiety bound non-covalently to the LIGCF surface. In currently preferred embodiments, the antibody is an anti-SARS-2-spike-protein, specifically binding to the spike proteins of covid-19-SARS-2 virions.

The targeting moiety may sometimes be directly bound to the LIGCF, particularly when LIGCF contains an appreciable amount of edge hydroxyls. At times, however, it may be beneficial to bind the targeting moiety via a linking moiety. The linking moiety may be a single molecule or may be a cascade of intermediate molecules readily connecting to one another, and possessing at least one function that is capable of interacting with LIG-like materials, and at least one function capable of binding a targeting moiety. The linking moiety may thus facilitate or even enable the binding of the targeting moiety onto the LIGCF flake. For example, the linking moiety may comprise a complex of 1-pyrenebutyric acid (PBA), in which the acid is transformed to an active ester using EDC and sulfo-NHS. The compound 1-pyrenebutyric acid binds to LIG via hydrophobic interactions, and pi-pi interaction and contains a carboxylic acid function. The latter can covalently bind to antibodies via a classic EDC-sulfo-NHS chemistry. EDC is 1-ethyl-3-(3-dimethylamino) propyl carbodiimide, usually provided as hydrochloride salt, and sulfo-NHS is N-hydroxysulfosuccinimide. EDC may also be used alone, requiring quenching after binding of a primary-amine-containing protein, e.g. with mercaptoethanol. In presence of sulfo-NHS EDC binds the antibodies (usually comprising several tens of available lysine residues on the Fc domain) to the carboxyl group of the pyrenebutyric acid.

The LIGCF coated with a targeting moiety may also be coated with a binding-blocking moieties, to reduce non-specific binding, e.g., to the uncoated portions of the LIGCF, e.g., by adsorption of specific or non-specific molecules. For example, after functionalizing with an antibody, the functionalized LIGCF may be incubated with bovine serum albumin.

Thus, the method of manufacturing of functionalized LIGCF may comprise providing a macroscopic LIGCF, optionally trimmed to a desired shape and dimensions, and exposing it consecutively to a linking moiety, to a targeting moiety, and optionally to a binding-blocking moiety. Exposing to the linking moiety may comprise the sub-steps of exposing LIGCF to a substance reactive or interacting with LIG-like materials, the substance further comprising at least one functional group not reactive or interacting with LIG-like materials, exposing the LIGCF exposed to the first substance to a consecutive substance(s) reactive with the functional group which is not reactive with LIG-like materials, to obtain a partially functionalized LIGCF, and exposing the partially functionalized LIGCF to a targeting moiety, comprising a group reactive with functional groups obtained on the partially functionalized LIGCF.

The functionalizing step(s) may usually be performed in a liquid medium, e.g., in an aqueous medium, such as phosphate-buffered saline solutions. The final LIGCF may then require drying before further processing. The drying may be performed in vacuo, and may also include lyophilizing the LIGCF, to preserve their functionalization moieties.

The macroscopic functionalized or non-functionalized LIGCF may be implemented in sensors. The LIGCF may be cut into a shape of a suspended cantilever beam. Preferably, the functionalization of the LIGCF should be performed after the forming of cantilever beam. For example, for a resonator sensor, the obtained functionalized cantilever beams may be affixed to a suitable surface, and suspended over a conductive electrode. Thus, this structure forms a mechanism in which the suspended LIGCF and the conductive electrode operate as the moveable and stationary electrodes. By applying time-dependent voltage between the electrodes, an electrostatic force is induced, which can be used as the actuation force of the suspended LIGCF. The voltage applied to the sensor should be sufficient for the actuating the cantilever, and may be e.g., between 2 and about 50 Volts, preferably between about 10 and about 25 Volts. The sensor may then be calibrated by wetting the cantilever with a blank testing medium, and drying it at pre-determined conditions and time, e.g., at 32° C. for 20 minutes. The resonance amplitude of the cantilever is then determined, e.g., by dynamically actuating around their approximate resonance frequency, which is typically in a few Kilohertz range, and stored for future reference. Notably, the resonance frequency and actuation voltage are derived from the geometry of the freestanding LIGCF and its material constants, such as its Young's modulus, therefore the mechanical integrity and homogeneity of the graphene foam sheets as described herein actually enable for the first time to use these graphenic materials in macroscopic sensing applications, as described in greater detail in the appended examples. Notably, the electrostatic force also influences the resonance frequency, as high actuation voltage reduces the resonance frequency. However, the electrostatic force has, in most cases, subtle effect on the resonance frequency. On the other hand, an added mass that binds to the resonating LIGCF (e.g., target sensing material) has a significant influence over its resonance frequency. This principle may be utilized, by specific binding of the target molecules to the resonating LIGCF, thereby increasing their mass, and consequently, the resonance characteristics.

Thereafter, a test may be performed, e.g. by exposing the sensor to the test specimen, e.g., a solution, an exhaled air stream, an aerosol, and the like, followed by washing and drying for sufficient time. The sensor may be briefly washed and dried, e.g., at elevated temperature, to remove potentially adsorbed contaminants. The resonance amplitude of the cantilever may then be determined again and compared to the reference. If the test specimen comprises the target molecules, the frequency response will include a shift to a different frequency as compared to the cantilever with unbound target molecules, and with a various resonance amplitude.

The preferred embodiments and the drawings provided herein demonstrating some of the embodiments of the present invention are provided to better understand the present invention, which however does not limit the invention in any respect. Many variants and equivalents may be readily envisaged by the skilled artisan; the invention therefore encompasses all these variations and equivalents. All scientific and technical terms used herein have meanings commonly used in the art unless otherwise specified. The definitions provided herein are to facilitate understanding of certain terms used frequently herein and are not meant to limit the scope of the present disclosure. As used herein the term “about” refers to ±10%. The terms “comprises”, “comprising”, “includes”, “including”, “having” and their conjugates mean “including but not limited to”. This term encompasses the terms “consisting of” and “consisting essentially of”, which have their narrower meaning as known in the art. It is appreciated that certain features of the invention, which are, for brevity, described in the context of separate embodiments, may also be provided in combination in a single embodiment. Conversely, combinations of various features of the invention, which are, for clarity and demonstration, described in the context of a single embodiment, may also be provided separately or in any suitable sub-combination, or as suitable in any other described embodiment of the invention. It must be noted that, as used in this specification and the appended claims, the singular forms “a”, “an” and “the” include plural referents unless the content clearly dictates otherwise. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C). Certain features described in the context of various embodiments are not to be considered essential features of those embodiments, unless the embodiment is inoperative without those elements.

### EXAMPLES

### Example 1—LIG Continuous Flakes

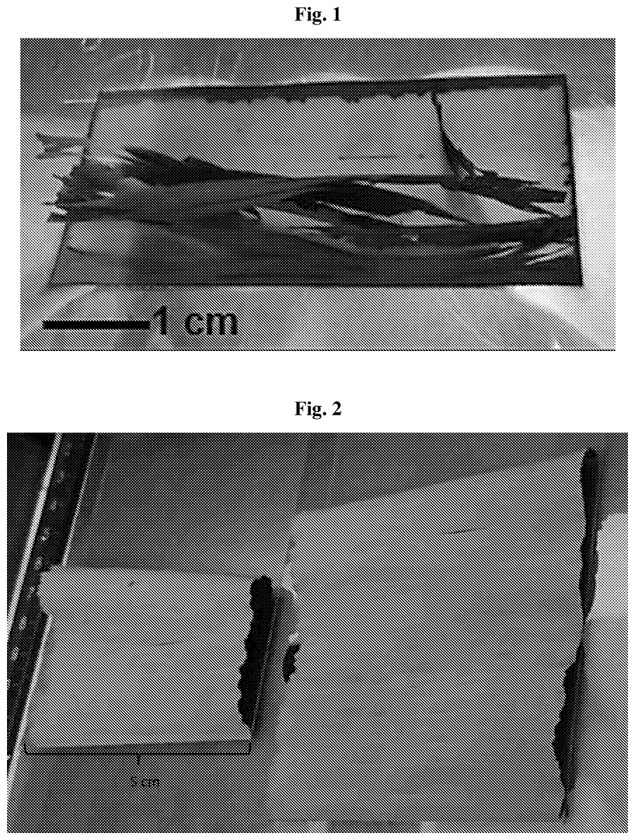

The following method was used for the generation of small freestanding graphene flakes: Commercial Polyimide (PI) solid sheet film with thickness 127 μm was attached onto the stage of a Universal Laser System (VLS 3.50), equipped with a 10.6 μm CO₂ pulsed laser (50 W) with 50.08 mm focal length lens (having a focal spot diameter of 130 microns) using an adhesive tape. Laser parameters were power (13%), laser scanning rate (18%), and image density of 1000 PPI with still air as the atmosphere (FIG. 1). Air was blown at the lens to keep the lens free from possible dust contamination. The first cycle of lasing produced freestanding LIG flakes with no or very weak attachment with the PI surface (FIG. 1).

The same procedure was repeated without removing the previously lased substrate with the same parameters without changing the focal distance to the same PI surface. This second laser treatment produced additional amount of freestanding LIG flakes, also with negligible attachment with the PI substrate surface.

The obtained LIGCF were of acceptable quality and mechanical properties to be further processed into resonator cantilever beam.

### Example 2—Laser Parameters for Freestanding LIGCF Formation

The same 10.6 μm CO₂ laser system, with the power source of 50 W was used. Optimization of laser parameters for LIG flakes formation was performed. During the optimization of laser parameters, the laser was used in Ras/vect mode, engraving field units were kept to metric, laser image density was kept to 6. To the laser platform air was blowing toward the laser lens to prevent debris from contaminating the lens. The relative humidity was 46.1±0.8%, and the laser environment temperature was 23.9±1.5° C. The exhaust from the laser system was connected to a fume hood to remove any gases formed during the lasing process.

First, the laser focus effect has been examined. The offset of the z-axis was changed between −0.7 mm and +0.7 mm, on a sample of 1×10 cm², at 13% of laser power, 18% of laser scanning speed, and 1000 pulse per inch density. It can be seen that the z-offset of between (−0.7) mm and (−0.2) mm produced LIGCF, whereas in other cases a coating of various degree of uniformity was obtained on the polymer. This offset corresponds to 0.1 mm-0.6 mm shift of focal distance of the lens away from the laser, respectively.

Then, at 13% of laser power, 18% of laser scanning speed, 1000 pulse per inch, and −0.4 mm z-offset, the minimum laser exposure area was determined by lasing areas between 1 and 5 square centimeters. It became readily evident that the size of the LIGCF is dependent on the exposure area, and that exposing areas of less than 1 cm² will probably lead to poor outcome.

Thereafter, the output laser power was evaluated at the same conditions to define the working parameters. The laser power was changed between 7 and 20%, i.e. between 3.5 and 10 W. It was observed that acceptable LIGCF were obtained at power output of between 4.5 and 9 W. The specific laser power was changed by 1-percent steps, between 7% and 20%.

Lastly, as laser power is one of the dominating parameters in the optimization of LIG flakes formation, laser scanning speed was evaluated. Using the same parameters, the scanning speed was changed between 12% and 24%. LIG flakes formation was best observed at the scanning speeds of between 14% to 22%.

On the basis of visual observations freestanding LIG flake formation is observed at the following operational parameters:
(a) By changing the laser platform in the z direction from its normal position (z=0), we can observe the formation of flakes in the range of (−0.7) mm to (−0.2) mm of platform shift
(b) For the formation of flakes a minimum size of laser treatment required is 1×4 cm², length and width of the flakes are highly dependent of the sample size.
(c) For a 50 W laser system flakes formation can be observed in the power range of 9% to 18% laser power, which corresponds to 4.5 W to 9 W of laser power consumption.

### Example 3—Extra-Large Standalone Continuous LIG Flakes (LIG Sheets)

Larger platform offsets beyond the apparatus capabilities were investigated. From zero platform position (laser-focused), the substrate film was mounted on either one microscope slide (1 mm thick), or 2 microscope slides stacked on one another (2 mm thick) to allow the substrate being moved towards the laser for larger offsets.

Freestanding laser-induced graphene continuous sheets obtained.

A Universal Laser systems (VLS 3.50) 50 W CO₂ laser system was used with a lens rated to be focused at a distance of 50.8 mm (2 inches, having a focal spot diameter of 130 microns) from the substrate. As mentioned above, to bring the platform into a focus-offset position, a polyimide (PI) film (˜127 micron thick) was mounted directly on the platform and the top of the substrate was adjusted to a focused position with the supplied focusing tool. From this zero platform position (focused), a PI film mounted on either one microscope slide (1 mm thick), or 2 microscope slides stacked on one another (2 mm thick) was placed on the laser platform. The platform was then moved toward the laser lens. The freestanding LIG could be achieved when the platform was moved 1.2 mm-1.7 mm towards the laser lens. Therefore, for example, if the substrate was added on 2 microscope slides plus moving the platform the maximum distance (1.7 mm), the total distance of the substrate moved toward the lens is 3.7 mm. Thus, the variation of tested settings included 1.2 mm-3.7 mm toward the laser lens, in which LIG sheets of various quality were obtained. The parameters were as follows: power: 12% of total laser power=6 W, laser scanning speed=30% of laser speed for corresponding laser parameters, laser pulse per inch=1000, image density=6, laser engraving mode=Rast/Vect.

Sheets of acceptable quality were obtained with several offsets. LIG sheets were obtained at offsets of between 98% and 92%, and the best quality sheets were obtained at offset of 47.1 mm of 50.8 mm, i.e., at about 92.7% of the focal length. Other excellent sheets were obtained with offset 48.1 mm/50.8 mm=94.7% of the focused position.

A representative photograph of the sheets is presented in FIG. 2. In the Figure, the sheets obtained at 92.7% offset were about coextensive with the laser irradiation area, which was about 50 square centimeters for the left-hand partially rolled sheet (about 5×10 cm), and about 96 square centimeters (8×12 cm) for the right-hand sheet. It is believed that further offset of up to about 85-90% may produce freestanding LIG sheets at similar parameters.

### Example 4—Characterization of LIGCF and LIG Sheets

The obtained freestanding flakes from the Example 2 and the LIG sheets from the example 3 were characterized with scanning electron microscopy (SEM) (FIGS. 3 and 4, respectively). SEM images were collected using a FEI JEOL IT 200. In the FIG. 3, the upper row of micrographs represents top view of the LIG continuous flake at varying magnification, with panels designated “(a)”, “(b)”, and “(c)” representing the increasing magnification as indicated by the reference bar in the right lower corner of each panel, representing 0.5 mm, 10 μm, and 5 μm, respectively. The flake is ca. 1.5 mm wide and is suitable for cantilever or resonator-like applications. The upper central and right panels “(b)” and “(c)” show the top view of LIG flakes-fiber, which has a typical 3D dense fibrous LIG structure. Micrographs at the middle row represent the bottom view of the same LIG freestanding flake, with panels designated “(d)”, “(e)”, and “(f)” representing the increasing magnification as indicated by the reference bar in the right lower corner of each panel, representing 20 μm, 10 μm, and 5 μm, respectively. The bottom micrograph is a side view of the flake, demonstrating homogenous essentially crack-free microstructure. The images of the bottom part of the LIG flake-fiber demonstrate the honeycomb-like porous microstructures of these freestanding LIG flakes. The average pore size of the large pores in the bottom of the flake was measured to be ca. 4 μm.

The Extra-large Freestanding Laser-induced graphene sheets obtained in the example 3 were also characterized by SEM (FIG. 4). It was observed that the macrostructure was different from that of the other flakes. A wave-like, or worm-like topology was observed. The upper row of micrographs represents top view of the extra-large LIG sheet (i.e., from the side of laser irradiation), at varying magnification representing the increasing magnification as indicated by the reference bar in the right lower corner of each inlet, representing 0.5 mm, 50 μm, and 20 μm, respectively. The bottom row follows the same magnification pattern from the bottom part of the sheet (i.e., the side that touched the substrate).

Raman spectroscopy was used for the determination of graphitic and crystalline structure of the obtained carbon material. A Horiba LabRam HR evolution micro-Raman system was used, equipped with a Synapse Open Electrode CCD detector air-cooled to −60° C. The excitation source was a 532 nm laser with a power of 0.5 mW. The obtained freestanding LIG flakes from the example 2 were characterized with Raman spectroscopy (FIG. 5). In the FIG. 5, Raman spectra for flakes obtained at varying laser power of the experiment described in the example 2 are presented, with the Raman shift being presented along abscissa axis, designated as “Raman Shift (1/cm)”, and the stacked spectra with the intensity (charted along the ordinate axis, designated as “Intensity (a.u.)”) measured for the varying laser powers, designated, from top to bottom, as “14%”, “13%”, “12%”, “11%”, “10%”, and “9%”, respectively.

Raman spectra of all freestanding LIG flakes demonstrated three typical peaks, similar to LIG that remains embedded in the substrate. On the basis of the corresponding peak intensities, it can be seen that the most prominent graphitic structure was obtained when laser was used with 12% of its total power, as described above, by using the parameters of scan speed of 18%, stage position z=−0.4 mm, image density 6, pulse per inch 1000. These optimum freestanding LIG flakes which I_{D}/I_{G}=0.47 shows the presence of defects similar to previously reported LIG embedded on a PI sheet. Presence of 2D peak in the spectra confirms the presence of multi-layered graphene in the freestanding LIG flakes (I_{2D}/I_{G}=0.592). These ratios ((I_{2D}/I_{G})/(I_{D}/I_{G})=1.26) show the presence of certain amounts of defects.

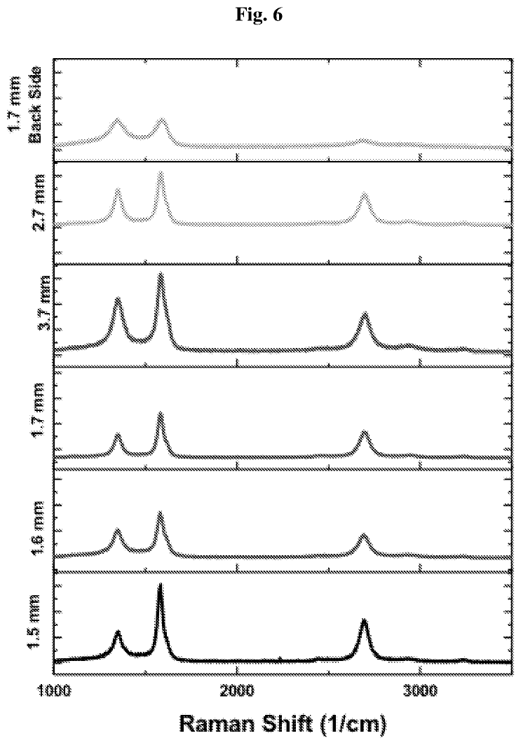

Raman analysis of the extra-large LIG sheets, as presented in FIG. 6, displayed the same d, G and 2d peaks indicating graphene content. In the FIG. 6, Raman shift is presented along abscissa axis, designated as “Raman Shift (1/cm)”, and the stacked spectra with the intensity (charted along the ordinate axis, designated as “Intensity (a.u.)”) measured for the varying offsets, designated, from top to bottom, as “1.7 mm Back Side”, “2.7 mm”, “3.7 mm”, “1.7 mm”, “1.6 mm”, and “1.5 mm”, respectively. It is observed that Raman signals on the top side and the bottom side are not exactly the same in the extra-large LIG sheets obtained in the example 3, indicating that the graphitic content is different on the top surface compared to the bottom surface.

Raman spectrum of the LIGCF obtained at currently preferred manufacturing conditions (at 12% laser power) is presented separately in FIG. 7. In the FIG. 7, Raman shift is presented along abscissa axis, designated as “Raman Shift (1/cm)”, and the intensity charted along the ordinate axis, designated as “Intensity (a.u.)”. The caption “12%-” designates the laser intensity setting.

To further quantify the chemical composition of freestanding LIG flakes obtained in the Example 2, XPS spectroscopic analysis were carried out. The high-resolution spectrum had only 3 dominant peaks for carbon, oxygen and nitrogen with atomic composition of 94.64%, 4.37%, and 0.99%, respectively. This chemical composition is similar to embedded LIG. A detailed deconvolution of Cl core level was further studied. This spectrum was de-convoluted, and two peaks were seen, one for sp²-hybridized C═C and another for hydroxyl and epoxy groups (C—OH and C—O—C).

### Example 5—Cantilever Resonating Sensor Preparation and Characterization

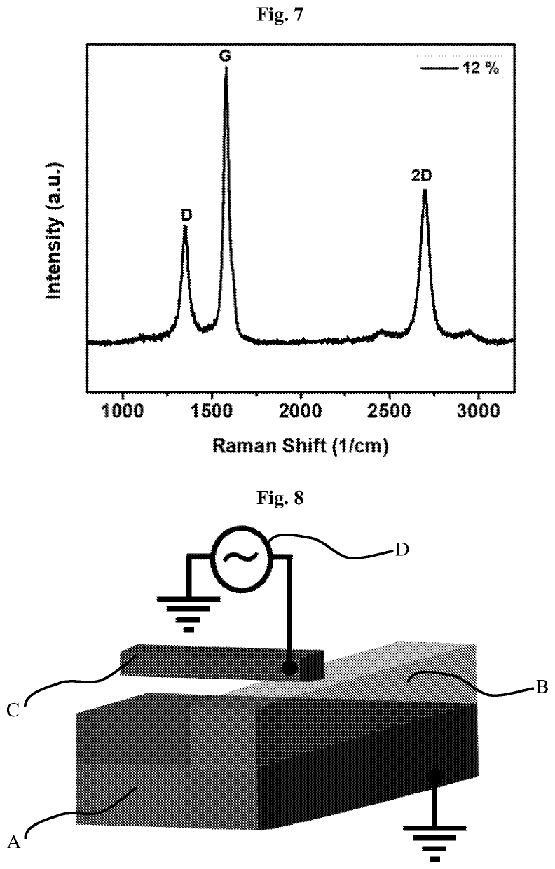

The LIG flakes were cut in small rectangular pieces and attached to a glass substrate over an earthed electrode using double-sided adhesive tape as a cantilever. Silver paste was added on the attached side of the LIG flake cantilever for an electrical connection point. Schematic representation of the resonating cantilever sensor is demonstrated in FIG. 8. In the FIG. 8, the earthed electrode A, is separated by the glass substrate B, whereon LIG flake cantilever C is attached. Time-dependent voltage D was applied when needed to the cantilever C, in the form of V (t)=V_{DC}+V_{AC} sin (2πft) (V_{DC} and V_{AC} are the DC and AC voltage components, respectively, f is the excitation frequency, and t is time). The voltage generator (network analyzer, as described below) was further earthed to close the circuit. The voltage between the suspended LIG flake cantilever C and the electrode A generates an electric field that induces an excitation electrostatic force. The excitation frequency was swept to acquire the frequency response of the devices.

The sensors were first washed in deionized water for 20 minutes, followed by 10 min drying in an oven set to 32° C. The electromechanical frequency resonance measurement was performed using LDV (Polytec MSA-500M) in conjunction with a network analyzer (Keysight E5061B) under ambient air conditions. The network analyzer provided a time-dependent voltage that generated an excitation force applied to the LIG flake. The excitation frequency was swept, while the LDV was pointed to the free end of the cantilever and captured the vibrations. The LDV readings were sent back to the network analyzer that acquired the frequency response. A Lorentzian function was fitted to the acquired response, from which extracted the resonance frequency and the quality factor.

The governing equation of motion was solved by first normalizing it to the non-dimensional form and then numerically solving using Runge-Kutta scheme implemented in MATALB software (ODE45 solver) to obtain the time response of the devices. We solved the equation for different exaction frequencies and extracted the steady-state amplitude for each frequency, thus, evaluating the frequency response. We repeated this process for varying added mass densities, hence, acquiring the relationship between the resonance frequency and the added mass.

The model of governing equation of motion of LIG flake cantilever and its boundary conditions were expressed as

$$EI \frac{{\partial }^{4} u}{\partial {x}^{4}} + C \frac{\partial u}{\partial t} + ρ A \frac{{\partial }^{2} u}{\partial {t}^{2}} = {f}_{ES} \qquad ( 1 )$$

$${{{{u \vert}_{x = 0} = 0 ; \frac{\partial u}{\partial x} \vert}_{x = 0} = 0 ; \frac{{\partial }^{2} u}{\partial {x}^{2}} \vert}_{x = L} = 0 ; \frac{{\partial }^{3} u}{\partial {x}^{3}} \vert}_{x = L} = 0 \qquad ( 2 )$$

where u is the deflection of the device, EI is the bending stiffness (E is Young's modulus and I is the mass moment of inertia), c is the damping coefficient, ρ is the mass density, and A is the cross section area of the cantilever. The boundary conditions (Equation 2) reflect zero deflection and zero angle at the clamped edge (x=0), and free end at x=L with zero shear force and bending moment.

To assess the mechanical stiffness of the devices, analyzing of the thermal vibrations of the cantilever was performed at 50° C. The equipartition theory states that under thermal equilibrium, the mean thermal energy equals the kinetic energy of the mean square of thermal vibrations, namely

$$\frac{1}{2} k {〈 z 〉}^{2} = \frac{1}{2} {K}_{B} T ,$$

where [symbol] z [symbol] ² is the means square thermal vibrations of the cantilever, k is the mechanical stiffness of the cantilever, K_{B} is the Blotzman factor, and T is the ambient temperature. The acquired thermal vibrations of several devices were then calculated the power density plot of the devices from the thermal vibrations. Finally, the stiffness was extracted as

$$k = \frac{{K}_{B} T}{{s}_{xx}} .$$

Here Sₓₓ is the area under the power spectrum density of the thermal vibrations. The stiffness of several devices showed bending stiffness values in the range of sub N/m.

The distributed excitation force was given as

$${f}_{ES} = \frac{{ε}_{0} {ε}_{r} w {V}^{2}}{2 {( {g}_{0} - u )}^{2}} \qquad ( 3 )$$

where ε₀≅8.854·10⁻¹² F/m is the dielectric permittivity of vacuum, εᵣ≅1 is the relative permittivity of air, w is the width of the cantilever, g₀ is the initial (undeformed) gap between the cantilever and the stationary electrode, and V is the excitation voltage. We solved the governing equation of motion using the Galerkin method with cantilever dimensions of 1.5 mm×0.5 mm×0.025 mm, initial gap g₀=7 mm, and initial mass density of ρ₀=90 kg/m³. The influence of the added mass was taken into our simulation by changing the LIG flake mass density such that ρ=ρ₀+Δp, where Δp is the added mass density. The simulated frequency responses for several values of added mass (calculated by Δm=ΔρV*, where V* is the volume of the cantilever) confirm that the added mass reduces the resonance frequency. Furthermore, the analysis indicated the frequency shift roughly corresponds to added masses of 0.32 ng, 0.40 ng, and 0.57 ng for concentrations of 56.5 nM, 96.0 nM, and 376.8 nM, for COVID-19 spike protein, respectively.

The mass sensing sensitivity is the ratio between the frequency shift, Δf, and the added mass, Δm, namely

$$S = \frac{Δ f}{Δ m} .$$

The model permitted extracting the sensitivity to be 1.06 kHz/ng which indicates the high sensitivity of the devices. Furthermore, the limit of detection (LoD) is an important parameter in the operation of the sensor, and it is defined as the added mass that corresponds to a frequency shift equal to three times of the detectable frequency shift, Δfₘᵢₙ, such that

$$LoD = \frac{3 Δ {f}_{min}}{S} .$$

In the present setup the network analyzer dictates the detectable frequency, which is estimated as Δfₘᵢₙ=2.81 Hz. Thus, the calculated LoD is 2.63 pg.

Since the mass of a single COVID-19 virion is estimated to be 524 ag, it has been concluded that under the present setup it can detect the binding of as few as 5,019 COVID-19 virus particles.

A variety of devices were produced with cantilevers of varying dimensions. The excitation frequency of the cantilevers was swept to acquire the frequency response. The frequency response of the cantilever sensors demonstrated an amplification of the vibrations around a frequency of between 0.5 and 10 kHz, depending on the length of the cantilever. The fundamental resonance frequencies of several devices as a function of their lengths demonstrate operation in the kilo-Hertz range with shorter devices showing higher resonance frequencies, within the tested length range of between about 1 mm and 2 mm. Thus, by simply shaping LIG flake cantilever devices with shorter lengths, higher frequencies can be achieved.

Additionally, increasing the voltage also increased the excitation force and the vibration amplitude, the latter in almost linear manner between 2 and 10 Volts, changing almost threefold. The resonance frequency, which is the frequency corresponding to the maximal amplitude, was also mildly modulated through the excitation voltage. Without being bound by a particular theory, this resonance frequency modulation was attributed to the softening effect of the electrostatic excitation force that reduces the stiffness of the devices, i.e., operating as a negative spring. Therefore, both frequency and amplitude could be tailored, which enables sensitivity control and greater flexibility in their operation.

### Example 6—Functionalization of Macroscopic LIGCF

Modification of graphene flakes for functionalized resonator sensor was performed as follows. LIG flakes obtained as in the example 1 were carefully placed in a 20 ml glass vial. First, 1-pyrenebutyric acid (PBA, CAS no. A17760, Alfa Aesar, Israel) solution, 10 mM in N-methyl-2-pyrrolidone (NMP CAS no. 5575-4100, Daejung chemicals and metals co. ltd., Korea) was added to the vial for 5 minutes, and thereafter the remaining PBA solution was removed with a micro-pipettor. Then, a solution containing EDC Catalog no. 25952-53-8, Chem-impex international, USA (5 mM) and sulfo-NHS Catalog no. 12831, Chem-impex international, USA (5 mM) in PBS butter was added and kept for 30 minutes. Then, the flakes were gently washed 3 times with deionized water. Afterwards, the flakes were incubated with 1:100 diluted anti-spike antibody (SARS-CoV-2 (2019-nCoV) Spike RBD Antibody, Rabbit PAb, Antigen, Affinity Purified, (Sino bio), supplied by: Enco scientific services, Israel) solution prepared in 0.01 M PBS buffer (pH=7.45) for 3 h at 4° C. The solution was removed and finally, a solution of 1% BSA CAS no. A2153-50G, Sigma-Aldrich, Israel in PBS buffer was added for 5 minutes onto the antibody-modified flakes to block the remaining surface to minimize non-specific interactions. Then, the flakes were gently washed 3 times with PBS buffer and kept overnight at 4° C. to maintain the integrity of antibody before they were used for further measurements and modifications, or alternatively freeze dried.

### Example 7—Sensing COVID-19 Spike Protein in Solution

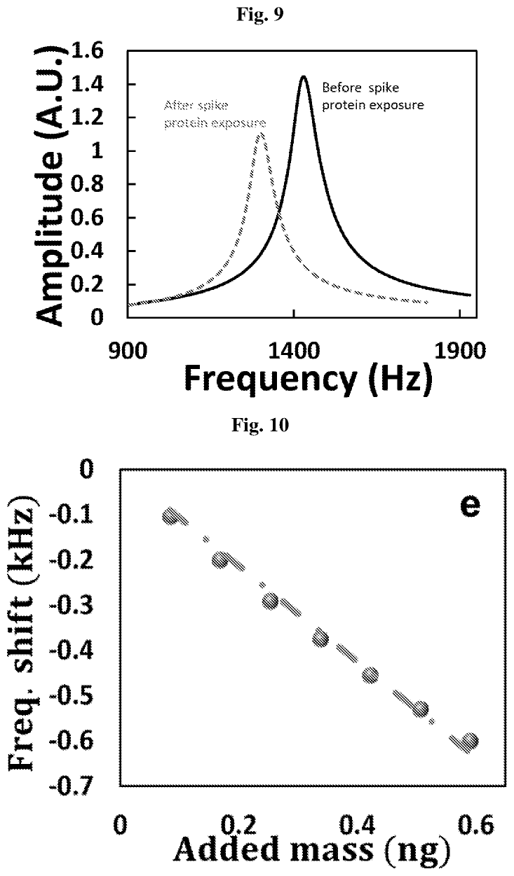

Freeze dried spike antibody loaded LIGCF prepared according to the example 6 were shaped as an approximately 1.5-mm cantilever and fixed onto a glass slide with silver paste, which also created an electrical connection. The suspended LIGCF devices were wetted and dried (in an oven for 10 minutes at 32° C.) several times, to ensure that the wetting of the suspended LIGCF alters its atomic structure and thus changing its resonance frequency, before the test. The resonance was detected by measuring the amplitude as function of the applied frequency, as escribed above. The results are presented in FIG. 9. In the FIG. 9, the amplitude is charted along the ordinate axis (designated as “Amplitude (A.U.)”), and the frequency is charted along the abscissa axis (designated as “Frequency (Hz)”).

Applying several wetting and drying cycles ensures that the atomic structure of the LIGCF stabilizes and frequency changes can be attributed only to added mass. Then, the resonators were dipped in PBS buffer, and oven dried for 20 minutes at 32° C., and characterized for frequency response (solid black line in FIG. 9).

Thereafter, different concentrations of spike protein were prepared in PBS (SARS-CoV-2 (2019-nCoV) Spike RBD-His Recombinant, Protein, (Sino bio), supplied by: Enco scientific services, Israel). The cantilever was then dipped in a solution of COVID-19 spike protein (96 nM) and oven dried for 1 hour at 32° C., in which the spike protein bound to the cantilever, followed by dipping the cantilever in the buffer to remove contaminations, and drying at 60° C. for 2 minutes. Then, the sensors were characterized again for frequency response. An example of shifts in frequency response are shown as the dashed gray line in FIG. 9.

A further experiment was conducted with about 1-mm long cantilevers, at applied voltage of 10-30V.

The concentrations of 56.5 nM, 96 nM, and 376.8 nM of the spike protein have produced frequency shifts of about 0.2, 0.5, and 0.6 kHz, respectively. Moreover, the addition of between 80 and 590 μg of the spike protein resulted in an almost linear reduction of the resonance frequency. The resultant correlation is presented in FIG. 10. In the FIG. 10, the negative frequency shift is charted along the ordinate axis, denoted as “Freq. shift (kHz)” versus the calculated added mass in nanograms, charted on the abscissa axis, denoted as “Added mass (ng)”. Thus, the described LIG flake devices can indicate both the existence of COVID-19 antigens, and also quantify the number of COVID-19 viruses present in the sample. This information may be very valuable providing insights on the stage and severity of the disease, as well and the level of contagion of a patient.

### Example 8—Joule Heating with LIG Sheets

Freestanding extra-large LIG sheets were conductively connected to a power source. Infra-red camera was placed vertically over the LIG sheet to monitor the temperature. Current was measured by a conventional amperemeter.

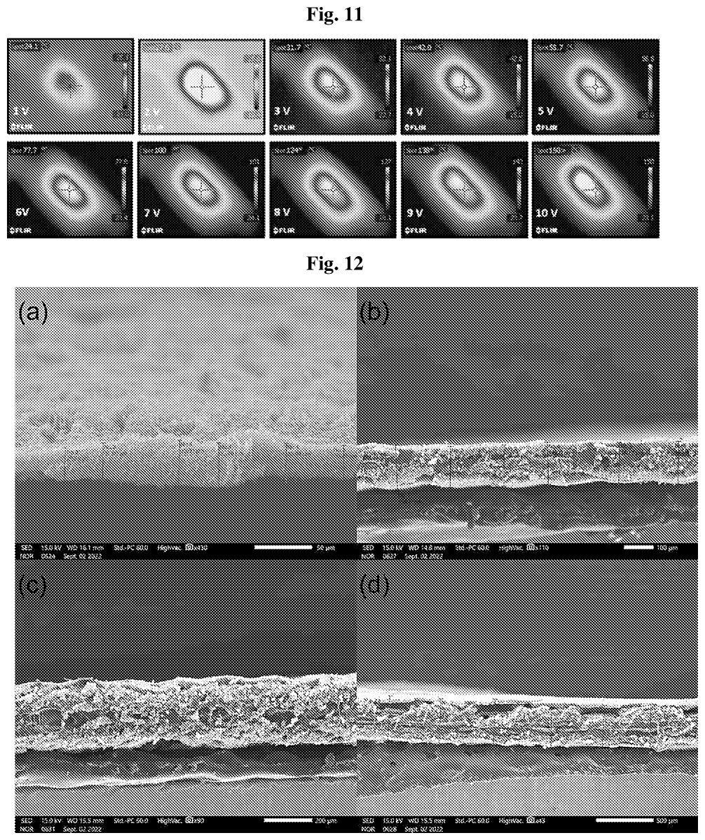

At applied voltage of below 1 Volt, low current was obtained, and the temperature remained about 27.6° C. The current increased linearly up to about 1.2 A at 10 V. These results indicate that LIG sheets are highly electrically conductive. Surface Joule heating were observed up to 150° C. for applied voltages of 10 V instantaneously. The greyscale thermal image captured by the camera is demonstrated in FIG. 11, with the series of images representing a specific voltage applied (designated as number plus V sign), and the temperature of the spot appearing in the upper left corner of each image.

### Example 9—LIG Sheets Composites

LIG sheet composites have been prepared by interlayering perforated tissue paper and LIG sheets. The tissue paper sheets included a pressure-sensitive adhesive to bond the LIG sheets. Perforations were included to facilitate the conductivity between the LIG sheets.

FIG. 12 demonstrates a SE micrograph of the layers. In the Figure, upper left panel (designated “a”) demonstrates the LIG sheet as obtained in the Example 3. The average diameter is about 35 μm, upper right panel (designated “b”) demonstrates one perforated tissue paper sandwiched in between two LIG sheets, with the average thickness of about 150 μm, lower left panel (designated “c”) demonstrates stacked two perforated tissue paper sandwiched in between three LIG sheets with the average thickness of 245 μm, and right bottom panel (designated “d”) demonstrates stacked three perforated tissue paper sandwiched in between four LIG sheets, with average thickness of about 390 μm.

1. A standalone macroscopic graphene foam sheet flake, wherein said graphene foam is laser-induced graphene, wherein said standalone sheet flake is not supported on a carrier or embedded in a polymer, wherein said standalone sheet flake is mechanically integral, and wherein said flake has a thickness of between 5 and 200 microns.

2. The flake according to claim 1, characterized by a porous stochastic morphology on one side, and a honeycomb-like cellular structure of a plurality of interconnected cells having a plan projection of a symmetric or asymmetric polygon having between 3 and 10 sides on the opposing side, as seen under suitable magnification using scanning electron microscopy (SEM).

3. The flake according to claim 2, wherein said honeycomb-like cellular structure has an overall appearance of co-oriented fused laser-induced graphene continuous-flake fibers.

4. The flake according to claim 2, wherein said honeycomb-like cellular structure has an overall appearance of an essentially uniform worm-like patterned laser-induced graphene continuous flake sheet with randomly oriented interfused laser-induced graphene continuous-flake fibers.

5. The flake according to claim 1, wherein said flake has a thickness of between 20 and 40 microns.

6. The flake according to claim 1, wherein said macroscopic flake has a length of at least 1 millimeter.

7. The flake according claim 6, wherein said length of at least 10 millimeters.

8. The flake according to claim 1, wherein said flake is mechanically integral over an area of between 1 square centimeter and 1,000 square centimeters.

9. The flake according to claim 1, further comprising a targeting moiety associated therewith.

10. The flake according to claim 7, wherein said targeting moiety is an anti-SARS-CoV-2 spike receptor-binding domain antibody.

11. A resonating sensor comprising a standalone macroscopic graphene foam sheet flake according to claim 1.

12. Method of manufacturing of standalone macroscopic graphene foam sheet flakes as defined in claim 1, comprising providing a sheet of a polymer, and irradiating said sheet of polymer with laser having power or energy density equivalent to that obtainable with laser power output of 4.5 to 9 W from a laser equipped with a lens providing a pulse diameter of 130 microns at 1000 pulses per inch density, wherein a focal offset of said laser is between about 100.2% and 101.2% of the focal length of said laser lens, or between 98% and 90% of the focal length of said lens, and wherein a minimum irradiation area of said polymer between 1 and 4 square centimeters.

13. The method according to claim 12, wherein said polymer is a polyimide.

14. The method according to claim 12, wherein said irradiation area is between 1 square centimeter and 1 square meter.

15. The method according to claim 14, wherein said focal offset of said laser is 90 and 98% of said focal length of said lens, and wherein said irradiation area is between 40 and 200 square centimeters.
